(19) United States
(12) Patent Application Publication
(10) Pub. No.: US 2010/0303920 A1
(43) Pub. Date: Dec. 2, 2010

(54) Aqueous Film Coating Composition / 841

(76) Inventor: Johan Hjartstam, Molndal (SE)

Correspondence Address: Pepper Hamilton LLP, 400 Berwyn Park, 899 Cassatt Road, Berwyn, PA, 19312-1183, US

(21) Appl. No.: 12/620,168

(22) Filed: Nov. 17, 2009

Related U.S. Application Data
(63) Continuation-in-part of application No. PCT/SE2009/050603, filed on May 27, 2009.

(51) Int. Cl. A61K 47/32; A61K 9/14

(52) U.S. Cl. 424/497; 514/772.2

(57) ABSTRACT

The invention relates to a film coating composition for use in coating a pharmaceutical formulation, said composition comprising a dispersion which comprises an acrylic polymer; a surfactant containing repeating ethoxy groups; a water-containing liquid; and a polyvinyl alcohol-polyethylene glycol graft copolymer.

### TECHNICAL FIELD

The present invention relates to an aqueous film coating composition for use in coating a pharmaceutical formulation. Furthermore, the invention also relates to a modified release formulation.

### BACKGROUND

In the pharmaceutical industry, polymeric film coating is an often utilised process in the manufacturing of pharmaceutical formulations. One example is multiple unit pellet formulations wherein each pellet is coated with an extended release film, such as a film comprising ethyl cellulose and hydroxypropyl methylcellulose. Organic solvents are often used in such coating processes, but should for environmental reasons be exchanged with water-based film forming materials.

Water-based film-forming polymer latexes for the pharmaceutical industry have been known since the early eighties to ninties when commercial dispersions more frequently appeared on the market, for instance dispersions sold under the trademarks Aquacoat®, Eudragit® and Kollicoat®.

One of the more interesting dispersions, due to the low glass transition temperature, is Eudragit® NM30D, which contains approximately 30% (˜28.5-31.5) w/w particles of the copolymer poly(ethylacrylate-co-methylmethacrylate) and approximately 0.7% w/w Macrogol stearyl ether (20) as emulsifier. Macrogol stearyl ether is obtained by ethoxylation of stearyl alcohol. Macrogol stearyl ether (20) has about 20 units (nominal value) of ethylene oxide per molecule.

The former dispersion Eudragit® NE30D contained the same polymer as Eudragit® NM30D but approximately 1.5% w/w of Nonoxynol 100 as emulsifier. To obtain satisfactory spraying conditions and technical properties of the film coat, an antisticking agent should be added to this polymer dispersion as reported by Petereit and Weisbrod 1995. Examples of antisticking agents utilised in connection with film forming dispersions are glycerol monostearate (GMS), talc and silica. It is often required to first disperse these substances with for instance a surfactant in order to obtain a more homogeneous system.

It is known that low molecular weight compounds, such as stabilizers, added to the dispersion can migrate in the film resulting in the film coat exhibiting a change in its properties, such as drug release profile, with time. Thus, it is desired to minimise the addition of extra additives to said polymer dispersion.

An exemplary film coating composition disclosed in WO 02/058677 (by AstraZeneca AB) comprises Eudragit® NE30D and sodium stearyl fumarate (sodium salt of 2-butanedioic monooctadecyl ester, PRUV™).

Moreover, in order to produce a film coat with enhanced water penetration and thereby providing an increased drug release rate, Eudragit® NM30D (or Eudragit® NE30D) may be mixed with a component having a higher solubility in water than said poly(ethylacrylate-co-methylmethacrylate).

An exemplary film coating composition disclosed in WO 03/051340 (by is AstraZeneca AB) comprises Eudragit® NE30D and Kollicoat® SR30D (containing approximately 27% w/w polyvinylacetate and approximately 2.7% w/w polyvinyl-pyrrolidone and 0.3% w/w sodium dodecylsulfate as stabilizers).

US 2005/0287211 relates to an oral pharmaceutical composition in particle form, which comprises particles that contain a drug at the core; a middle layer that contains two types of water-soluble components, an insolubilizer and an insolubilizing substance; and an outer layer for controlling water penetration that contains a water-insoluble substance and optionally one or more water-soluble substances. Several examples of both water-soluble substances and water-insoluble substance are given.

### SUMMARY OF THE INVENTION

An object of the invention is to provide an aqueous film coating composition for use in coating a pharmaceutical formulation to provide modified release of a pharmacologically active ingredient, where the resulting film coat preserves its physical properties upon storage.

According to a first aspect of the invention, this and other objects are achieved with a film coating composition comprising a dispersion which comprises: an acrylic polymer;

a surfactant containing repeating ethoxy groups;

a polyvinyl alcohol-polyethylene glycol graft copolymer; and

a water-containing liquid.

In an embodiment of the composition according to the invention, the acrylic polymer and the surfactant are provided by Eudragit® NE30D and the polyvinyl alcohol-polyethylene glycol graft copolymer is provided by Kollicoat® IR.

In a further embodiment, the composition according to the invention further comprises sodium stearyl fumarate.

According to a second aspect, the invention provides a film coat covering a pharmaceutical core which comprises a pharmacologically active ingredient, wherein the film coat comprises an acrylic polymer; a surfactant containing repeating ethoxy groups; and a polyvinyl alcohol-polyethylene glycol graft copolymer; and wherein the film coat has been deposited from a water-containing liquid.

According to a third aspect, the invention provides a pharmaceutical formulation comprising a pharmaceutical core comprising a pharmacologically active ingredient and the above-mentioned film coat applied thereon. Said formulation may be provided in a plurality of beads wherein each bead is coated with said film coat.

Other features and advantages of the present invention will become apparent from the following description of the invention.

### DETAILED DESCRIPTION OF THE INVENTION

The invention relates to a pharmaceutical film coating composition comprising:

an acrylic polymer;

a surfactant containing repeating ethoxy groups;

a polyvinyl alcohol-polyethylene glycol graft copolymer; and

a water-containing liquid.

In the Examples described hereinafter, it is shown that a film coat obtained from this composition preserves its drug release properties upon storage.

Without being bound by any theory, it is believed that the chemical similarities between the ethoxy groups of the surfactant and the polyvinyl alcohol-polyethylene glycol graft copolymer may reduce migration of the surfactant in the resulting film coat. The composition according to the invention thus provides a film coat which upon storage preserves its physical properties.

The new film coating system provides a minimal addition of extra additives to the dispersion before the film forming process, no aggregation of the dispersion, non-stickiness during processing, satisfactory mechanical strength of the resulting film coat, and reproducibility.

In particular, the acrylic polymer is an ethylacrylate/methylmethacrylate copolymer.

In particular, the surfactant contains within the range of from 5 to 20 repeating ethoxy groups. More particularly, the surfactant is a polyoxyethylated monoether, such as a Macrogol stearyl ether.

In a particular embodiment of the composition according to the invention, the acrylic polymer and the surfactant are provided by Eudragit® NM30D.

In a further particular embodiment of the composition according to the invention, the polyvinyl alcohol-polyethylene glycol graft copolymer is provided by Kollicoat® IR. The polymer of Kollicoat® IR consists of approximately 75% polyvinyl alcohol units and approximately 25% polyhethylene glycol units. Kollicoat® IR also contains approximately 0.3% colloidal silica.

The weight ratio of the acrylic polymer and the polyvinyl alcohol-polyethylene glycol graft copolymer is within the range from to 99.9/0.1 to 70/30.

In a further particular embodiment, the composition according to the invention comprises sodium stearyl fumarate. Sodium stearyl fumarate is available under the tradename PRUV®.

Suitably, the amount of sodium stearly fumarate in the film coating composition is in the range of from 0.05 to 15%, in particular from 2 to 10%, such as from 5 to 8% by weight.

Suitably, the water-containing liquid comprises water and a water-miscible organic liquid, for example lower alkanols such as ethanol, propanol or isopropanol. From a safety point of view it is advantageous that the proportion of the organic liquid is kept to a minimum but small amounts, such as in the range of 0 to 20% by volume, are tolerable. Preferably the liquid is water.

Further, the invention relates to a modified release pharmaceutical formulation which includes
a) a pharmaceutical core comprising a pharmacologically active ingredient; and
b) a film coat being applied on said core and comprising an acrylic polymer; a surfactant containing repeating ethoxy groups; and a polyvinyl alcohol-polyethylene glycol graft copolymer;
   wherein the film coat has been deposited from a water-containing liquid.

As used herein, the term “modified release formulation” means that the pharmacologically active ingredient is released over longer period of time, for example 1-20 hour(s) longer, than from an immediate release formulation.

In a further embodiment of the pharmaceutical formulation according to the invention, said formulation is in the form of a multiple unit formulation wherein each individual unit comprises a film coated pharmaceutical core as defined above.

Said multiple unit formulation may be in the form of a multiple unit tablet, capsule (such as capsules of hard gelatine or hydroxypropyl methylcellulose) or sachet, or as pellets or powder dispersed in a suitable container.

The individual units contained in such multiple unit formulations include microparticles, microspheres, microtablets, pellets and the like, and they do generally all comprise a core comprising the active ingredient. The core can be built on an inert starting seed on which is applied a layer comprising the active ingredient. The inert starting seed may be selected from particles of, for example microcrystalline cellulose (such as Celphere™ 203 or Celphere™ 305), silica or non-pareil. A suitable particle size range of the starting seed is, for example, between 0.15 and 0.30 mm or between 0.20 and 0.50 mm in diameter. The active ingredient may be applied onto the inert seed by using conventional techniques such as spray-crystallisation, spray-layering, rotor granulation, melt pelletisation, etc.

A modified release pharmaceutical formulation can also be obtained by mixing different types of units in the final dosage form of the multiple unit formulation, for example mixing a specific amount of units giving immediate release of the active ingredient with a specific amount of units providing an extended release of the active ingredient. The term “immediate release pellet” is in this case equivalent to an uncoated core as described above.

Thus, in one embodiment of the present invention there is provided a modified release pharmaceutical formulation in the form of a multiple unit formulation comprising:

I) immediate release units (e.g. pellets) comprising an active ingredient; and

II) extended release units (e.g. pellets) comprising an active ingredient, said units being coated a film coat as defined above.

The ratio between the pellets according to I and II may be in the range of from 10:90 to 90:10 (with respect to weight of active ingredient of the pellets according to I and II, respectively).

The active ingredient of the pharmaceutical formulation according to the invention may be the compound tert-butyl(2-{7-[2-(4-cyano-2-fluorophenoxy)ethyl]-9-oxa-3,7-diazabicyclo-[3.3.1]non-3-yl}ethyl)carbamate (hereinafter denoted as Compound A), or a pharmaceutically acceptable salt thereof.

The active ingredient may be present in form of a salt, such as acid addition salts. Examples of salts of Compound A are benzoic acid salts, hydroxy-substituted benzenesulphonic acid salts, 1- or 2-naphthalenesulphonic acid salts, 1,5-naphthalenedisulphonic acid salts, particularly, toluenesulphonic acid salts, or, especially, benzenesulphonic acid salts, methanesulphonic acid salts, hippuric acid salts, toluenesulphonic acid salts, pamoic acid salts, 1,5-naphthalenedisulphonic acid salts, terephthalic acid salts, succinic acid salts, or tartaric acid salts. Particular salts that may be mentioned include salts with fumaric acid or maleic acid.

Pharmaceutical formulations comprising an oxabispidine as active ingredient are previously known by WO 02/083687 and WO 02/083689. Formulations for modified release and for immediate release are described in these applications.

In one embodiment of the present invention, there is provided a modified release pharmaceutical formulation in the form of a multiple unit formulation comprising:

I) immediate release units (e.g. pellets) comprising Compound A, or a pharmaceutically acceptable salt thereof, and optionally activated charcoal; and II) extended release units (e.g. pellets) comprising Compound A, or a pharmaceutically acceptable salt thereof, said units being coated with one or more polymeric coating layers.

The ratio between the units according to I and II may be in the range of from 10:90 to 90:10, such as 10:90; 15:85; 20:80; 25:75; 30:70; 35:65; 40:60; 45:55; 50:50; 60:40; 70:30; 75:25; 80:20 and 90:10 (with respect to weight of active ingredient in the units according to I and II, respectively). More specifically, the ratio between the units according it, to I and II is within the range of from 30:70 to 35:65.

This specific formulation may be used in:
a) providing a therapeutic effect against cardiac arrhythmia primarily provided by the extended release pellets (II), and,
b) providing the possibility for early detection of patients responding with unfavourable QT-prolongation primarily provided by the immediate release pellets (I).

Suitable amounts of active ingredient in pharmaceutical formulation of the invention depend upon many factors, such as the nature of that ingredient (free base/salt etc), the dose that is required, and the nature and amounts of other ingredients in the formulation. However, the active ingredient may be in the range of from 0.5 to 80%, for example 1 to 75%, such as 3 to 70%, preferably 5 to 65%, more preferably 10 to 60% and especially 15 to 55% w/w.

The final dosage form of the pharmaceutical formulation of the invention may be dosed one or more times daily (e.g. up to six times, but preferably not more than twice daily), irrespective of the number of individual units (e.g. capsules, tablets) that are administered as part of one “dose”.

Typical daily doses of Compound A, or of pharmaceutically-acceptable salts thereof, are in the range 10 to 1000 mg of free base (i.e., in the case of a salt, excluding any weight resulting from the presence of a counter ion), irrespective of the number of formulations (e.g. tablets) that are administered during the course of that day. Examples of daily doses are in the range 20 to 1000 mg, such as 20 to 500 mg. A typical dose in a formulation according to the invention (e.g. a tablet) is thus in the range of from 15 to 500 mg, for example 20 to 400 mg. Doses of Compound A in the formulation may be such as 15, 20, 25, 30, 40, 50, 60, 70, 75, 80, 90, 100, 110, 120, 125, 130, 140, 150, 160, 170, 175, 180, 190, 200, 210, 220, 225, 230, 240, 250, 260, 270, 275, 280, 290, 300, 310, 320, 325, 330, 340, 350, 360, 370, 375, 380, 390 or 400 mg.

Formulations of the invention, whether in the form of a pellet system or otherwise, may contain one or more further excipients to further modify drug release, to improve the physical and/or chemical properties of the final formulation, and/or to facilitate the process of manufacture. Such excipients are conventional in the formulation of modified release formulations.

For example, formulations of the invention may contain one or more of the following diluents: calcium phosphate (monocalcium phosphate, dicalcium phosphate and tricalcium phosphate), lactose, microcrystalline cellulose, mannitol, sorbitol, titanium dioxide, aluminium silicate and the like. Preferred diluents include microcrystalline is cellulose.

Formulations of the invention may contain one or more of the following lubricants: magnesium stearate, sodium stearyl fumarate and the like.

Formulations of the invention may contain a glidant, such as colloidal silica. Formulations of the invention may contain one or more of the following binders: polyvinylpyrrolidone, lactose, mannitol, microcrystalline cellulose, a polyethylene glycol (PEG), pregelatinized starch, sucrose, maltodextrin, acacia, carbomers, sodium alginate, agar, gelatin, xanthan gum, polyvinyl alcohol (PVA), polyvinyl pyrrolidone (PVP), caragenaan, hydroxyethyl cellulose, a hydroxypropyl methylcellulose (HPMC) of a low molecular weight, a methyl cellulose (MC) of a low molecular weight, a hydroxypropyl cellulose (HPC) of a low molecular weight and the like.

Formulations of the invention may contain one or more of the following pH controlling agents: organic acids (e.g. citric acid and the like) or alkali metal (e.g. sodium) salts thereof, pharmacologically acceptable salts (e.g. sodium, magnesium or calcium salts) of inorganic acids (such as carbonic acid or phosphoric acid), oxides of magnesium, as well as alkali, and alkaline earth, metal (e.g. sodium, calcium, potassium and the like) sulphates, metabisulphates, propionates and sorbates.

Other further excipients may include colorants, flavourings, tonicity-modifying agents, coating agents, preservatives, etc.

Combinations of the above-mentioned further excipients may be employed. It will be appreciated by the skilled person that some of the above mentioned further excipients, which may be present in the pharmaceutical formulation of the invention, might have more than one of the above-stated functions.

The total amount of further excipients that may be present in the pharmaceutical formulation of the invention will depend upon the nature of the formulation, as well as the nature, and amounts of, the other constituents of that formulation, and may be an amount of up to 85%, for example between 0.1 to 75%, such as 0.2 to 65%, preferably 0.3 to 55%, more preferably 0.5 to 45% and especially 1 to 40%, such as 2 to 35% w/w of the total amount of excipient. In any event, the choice, and amount, of these excipient(s) may be determined routinely (i.e. without recourse to inventive input) by the skilled person.

The invention will now be illustrated by means of the following non-limiting examples.

In the Examples, Compound A is tert-butyl(2-{7-[2-(4-cyano-2-fluorophenoxy)-ethyl]-9-oxa-3,7-diazabicyclo-[3.3.1]non-3-yl}ethyl)carbamate.

### Examples 1-4

a) 5761 g of Compound A was dispersed and micronised in 11939 g of a 3.64% (w/w) solution of HPMC (6 cps) in water using a Silversson blender and a Dynomill.

b) 8571 g of the resulting suspension was then sprayed onto 400 g of microcrystalline cellulose spheres (Celphere 203, Asahi Casei) in a fluidised bed with subsequent drying (film yield 97%).

c) 100 g of the pellets were then coated with 150 g of polymer dispersion. The contents (w/w %) of the different polymer dispersions used are presented in the Table 1.

TABLE 1

| | Polymer dispersion (w/w %) | | | |
|---|---|---|---|---|
| | Example 1 | Example 2 | Example 3 | Example 4 |
| Eudragit ® NM30D | 44.9 | 44.3 | 43.7 | 44 |
| (dispersion) | | | | |
| Kollicoat ® IR | 2.3 | 3.4 | 4.6 | 5.6 |
| PRUV ® | 7.9 | 8 | 7.4 | 8 |
| Water | 44.9 | 44.3 | 43.7 | 44 |

The polymer dispersion was prepared by first adding PRUV to milli-Q water in a beaker. The mixture was heated to 60° C. and then stirred (at about 900 rpm) for approx. 2 minutes. The resulting suspension was then cooled down to room temperature before Kollicoat IR was added. This mixture of milli-Q water, PRUV and Kollicoat was then added to the Eudragit NM30D dispersion and set for stirring over the night with a stirring speed of 600 rpm before used in the film coating process.

Both the spray layering and the film coating were executed in a fluidised bed.

The spray layering of Compound A on the microcrystalline cellulose spheres was performed using Wurster process 1: 178 mm bottom plate diameter, Schlick 970-S3 spraygun with 1.2 mm liquid nozzle.

The coating step in which the polymeric film coat was applied was performed using is Wurster process 2: 120 mm bottom plate diameter, Schlick 970-0 spraygun with 0.8 mm liquid nozzle.

The relevant process parameters used for each step are given in Table 2 below.

TABLE 2

| | Wurster process 1 | Wurster process 2 |
|---|---|---|
| Parameter | (spray layering) | (polymeric coating) |
| Inlet air temp (° C.) | 80 | 30 |
| Outlet air temp (° C.) | 34 | 18 |
| Air flow (m³/h) | 130 | 35 |
| Atomizer pressure | 3.5 | 2.5 |
| (bar) | | |
| Liquid flow (g/min) | 71 | 5.5 |

A coalesced step was followed the coating process in order to stabilize the film properties. The pellets were then kept in a climate chamber at 60° C., 40% RH for about 52 hours. Alternatively, the pellets may be cured in the fluid bed at about 50-60° C. for about 1-2 hours.

### Release Profiles

The release of Compound A from pellets obtained in accordance with Examples 1-4 were measured by keeping the determined amount of pellets in a basket which was added to a dissolution bath of phosphate buffer solution having pH 6.8. An USP apparatus 2 (paddle at 50 rpm) was used. The amount of Compound A dissolved in the bath was determined by spectrophotometric detection (Agilent Dissolution testing system with an UV detector). The release profiles are presented in Table 3.

TABLE 3

% (w/w) of Compound A released

| Time (h) | Example 1 | Example 2 | Example 3 | Example 4 |
|---|---|---|---|---|
| 0 | 0 | 0 | 0 | 0 |
| 0.5 | 0.1 | 1.5 | 7.9 | 9.7 |
| 1 | 0.3 | 4.3 | 14 | 18 |
| 2 | 1.1 | 11 | 24 | 33 |
| 3 | 2.8 | 18 | 33 | 46 |
| 4 | 5.1 | 26 | 42 | 57 |
| 5 | 7.5 | 32 | 51 | 68 |
| 6 | 10 | 39 | 59 | 78 |
| 8 | 15 | 52 | 74 | 92 |
| 10 | 20 | 64 | 86 | 97 |
| 12 | 25 | 75 | 94 | 99 |
| 15 | 31 | 87 | 99 | 99 |
| 20 | 41 | 96 | 100 | 100 |
| 25 | 51 | 99 | 100 | 100 |

The pellets according to Example 3 was stored at 40° C., 75% RH for 8 weeks. The release profile after storage is presented in Table 4.

TABLE 4

| | Example 3 after 8 weeks storage |
|---|---|
| Time (h) | % (w/w) of Compound A released |
| 0 | 0 |
| 0.5 | 8.7 |
| 1 | 15 |
| 2 | 25 |
| 3 | 34 |
| 4 | 42 |
| 5 | 50 |
| 6 | 57 |
| 8 | 72 |
| 10 | 85 |
| 12 | 93 |
| 15 | 97 |
| 20 | 100 |
| 25 | 100 |

As shown in Table 4, the drug release profile is preserved after storage.

While the invention has been described in detail and with reference to specific embodiments thereof, it will be apparent for one skilled in the art that various changes and modifications can be made therein without departing from the spirit and scope thereof.

1. A film coating composition for use in coating a pharmaceutical formulation to provide modified release of a pharmacologically active ingredient, said composition comprising a dispersion which comprises:
a) an acrylic polymer;
b) a surfactant containing repeating ethoxy groups;
c) a water-containing liquid;
and characterized in further comprising:
d) a polyvinyl alcohol-polyethylene glycol graft copolymer.

2. A pharmaceutical film coating composition according to claim 1, in which the acrylic polymer is an ethylacrylate/methylmethacrylate copolymer.

3. A pharmaceutical film coating composition according to claim 1, in which the surfactant contains within the range of from 5 to 20 repeating ethoxy groups.

4. A pharmaceutical film coating composition according to claim 1, in which the surfactant is a polyoxyethylated monoether.

5. A pharmaceutical film coating composition according to claim 1, in which the surfactant a Macrogol stearyl ether.

5. A pharmaceutical film coating composition according to claim 1, in which the acrylic polymer and the surfactant are provided by Eudragit® NE30D.

6. A pharmaceutical film coating composition according to claim 1, in which the polyvinyl alcohol-polyethylene glycol graft copolymer is provided by Kollicoat® IR.

7. A pharmaceutical film coating composition according to claim 1, further comprising sodium stearyl fumarate.

8. A modified release pharmaceutical formulation which includes
a) a pharmaceutical core comprising a pharmacologically active ingredient; and
b) a film coat being applied on said core and comprising an acrylic polymer; a surfactant containing repeating ethoxy groups; and a polyvinyl alcohol-polyethylene glycol graft copolymer;
wherein the film coat has been deposited from a water-containing liquid.
